(19) United States
(12) Patent Application Publication
(10) Pub. No.: US 2025/0135183 A1
(43) Pub. Date: May 1, 2025

(54) HUBS FOR CATHETER BLOOD PUMPS

(71) Applicant: Shifamed Holdings, LLC, Campbell, CA (US)

(72) Inventors: Daniel VARGHAI, Campbell, CA (US); Michael CALOMENI, San Jose, CA (US); Brian D. BRANDT, Morgan Hill, CA (US); Seyed Mostafa GHOREYSHI, Campbell, CA (US); Daniel HILDEBRAND, Santa Cruz, CA (US); Janine ROBINSON, Half Moon Bay, CA (US)

(21) Appl. No.: 18/835,630

(22) PCT Filed: Feb. 2, 2023
(86) PCT No.: PCT/US2023/061871

§ 371 Date: Aug. 2, 2024

Related U.S. Application Data
(60) Provisional application No. 63/267,468, filed on Feb. 2, 2022.

(51) Int. Cl. A61M 60/81; A61M 60/122; A61M 60/216

(52) U.S. Cl. CPC A61M 60/81; A61M 60/122; A61M 60/216; A61M 2205/04; A61M 2205/103; A61M 2205/3331; A61M 2207/10

(57) ABSTRACT

Catheter blood pumps that include an expandable conduit coupled to at least one hub shaped to promote smooth blood flow. In some examples, the at least one hub includes a body with radially extending arms that are coupled to struts of the conduit. The hub may include channels having curved surfaces for promoting non-turbulent fluid flow around a distal opening and/or a proximal opening of the conduit. The arms may have ridge regions that extend axially along the body of the hub to define at least a portion of the channels.

### CROSS REFERENCE TO RELATED APPLICATIONS

This application claims the benefit of priority to U.S. Application No. 63/267,468, filed Feb. 2, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. Intra-aortic balloon pumps (IABP) are used to support circulatory function, such as treating heart failure patients. An IABP is typically placed within the aorta, and inflated and deflated in counter-pulsation fashion with the heart contractions, with one function being to provide additive support to the circulatory system. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

More recently, minimally invasive rotary blood pumps have been developed, which are inserted into the body in connection with the cardiovascular system to pump arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient. The smallest rotary blood pumps currently available may be percutaneously inserted into the vasculature of a patient through an access sheath, thereby avoiding more extensive surgical intervention, or through a vascular access graft. One such device is a percutaneously inserted ventricular support device.

Although blood pumps exist, there is a need to provide additional improvements in the field of ventricular support devices and similar blood pumps for treating compromised cardiac blood flow.

### SUMMARY OF THE DISCLOSURE

The disclosure is related to intravascular blood pump and methods of their use.

One aspect of the disclosure is a hub for an intravascular pump, comprising: a hub body; and a plurality of arms radially extending from the body, wherein the plurality of arms each have an opening sized and configured to receive a strut of a scaffold structure of the intravascular pump.

In this aspect, the plurality of arms may be comprised of a polymer material.

In this aspect, the body may be comprised of a polymer material.

In this aspect, the body and the plurality of arms may be comprised of a same polymer material.

In this aspect, the body and the plurality of arms may be comprised of different polymer materials.

In this aspect, the plurality of arms may be made of a stiffer polymer than the body.

In this aspect, the body may be integrally formed with the plurality of arms.

In this aspect, the hub may be formed by injection molding.

In this aspect, each of the arms may have a distal opening with engagement features configured to engage with a distal end of a corresponding strut.

In this aspect, the hub may be integrally formed with or bonded with a distal tip of the intravascular pump, the distal tip having an atraumatic distal end.

In this aspect, each of the plurality of arms may have a webbed base that extends axially along the body to define channels between adjacent arms.

In this aspect, the hub may further comprise a tapered feature extending proximally from the hub body. The webbed bases may transition from each of the plurality of arms to a tapered feature that extends proximally from the hub body.

In this aspect, the webbed bases of the plurality of arms may form channels along the body of the hub between the plurality of arms.

In this aspect, the body may have a lumen that run axially through the body. The lumen can extend axially from the tapered portion through the hub body.

In one aspect, the hub can further include a pressure sensor disposed within the hub body. The hub may further include at least one pressure senor wire routed proximally from the pressure sensor along at least one of the plurality of arms.

One aspect of the disclosure is an intravascular blood pump, comprising: a collapsible conduit having an inner lumen for passing fluid therethrough, the conduit comprising a proximal end having a proximal opening, and a distal end having a distal opening; at least one impeller within the conduit, the at least one impeller arranged to pump fluid into the distal opening of the conduit and out of the proximal opening of the conduit; a plurality of struts extending from the distal end of the conduit; and a hub comprising a hub body with a plurality of arms extending radially from the hub body, the plurality of arms each have an opening sized and configured to receive one of the plurality of struts.

In this aspect, the struts may be comprised of nitinol and the plurality of arms are comprised of a polymer material.

In this aspect, the plurality of arms may have ridge regions that extend axially along the body to define channels shaped to reduce flow separation, recirculation, and/or stagnation into the distal opening or out of the proximal opening of the conduit.

In this aspect, the ridge regions of the plurality of arms may be axially aligned with a central axis of the hub.

In this aspect, the channels may be longer in an axial direction than in a circumferential direction.

In this aspect, each of the plurality of arms may have a distal region that extends from a corresponding ridge region, wherein the distal region is coupled to a corresponding strut.

In this aspect, from a side perspective, the ridge region may be wider than the distal region.

In this aspect, from a front perspective, the distal region may be wider than the ridge region.

In this aspect, the distal region may gradually taper to the ridge region.

In this aspect, the body may be integrally formed with the plurality of arms.

In this aspect, the hub may be formed by injection molding.

In this aspect, the hub may be coupled to a distal tip of the intravascular blood pump.

In this aspect, the hub may be integrally formed with the distal tip.

In this aspect, each of the plurality of arms may have an opening in which a corresponding strut is secured within.

In this aspect, a distal end of each of the plurality of struts may have engagement features that enhance coupling with a corresponding arm.

In this aspect, each of the plurality of arms may be molded over a distal end of a corresponding strut.

In some aspects, a distal end of each of the plurality of struts terminates in the plurality of arms.

In other aspects, a distal end of each of the plurality of struts terminates in the hub body.

In this aspect, at least a portion of the plurality of arms may be sufficiently flexible to bend radially inward when the conduit is in a radially collapsed state.

In this aspect, the impeller is disposed within a proximal region of the conduit.

In this aspect, a proximal end of the conduit may include an impeller region having an impeller therein.

In this aspect, the hub may be distal to the conduit and may be configured to reduce flow separation, recirculation, and/or stagnation into the distal opening of the conduit.

In this aspect, the intravascular blood pump may include only one impeller.

In this aspect, the intravascular blood pump may include at least two impellers.

In one aspect, the intravascular blood pump may include a tapered feature extending proximally from the hub body.

In one aspect, the ridge regions transition from each of the plurality of arms to a tapered feature that extends proximally from the hub body.

In one aspect, the hub body has a lumen that extends axially through the hub body.

In another aspect, the hub body has a lumen extends axially from the tapered feature through the hub body.

In one aspect, the intravascular blood pump may include a pressure sensor disposed within the hub body.

In one aspect, the intravascular blood pump may include at least one pressure sensor wire routed proximally from the pressure sensor along at least one of the plurality of arms.

In one aspect, the intravascular blood pump may include a membrane formed on the collapsible conduit extending from the proximal opening to the distal opening.

In one aspect, the plurality of struts extend from a distal end of the membrane to the opening into the plurality of arms of the hub.

In one aspect, the intravascular blood pump may include a coating disposed over the plurality of struts.

In one aspect, the coating comprises a polymer coating.

In one aspect, the coating is configured to reduce or eliminate clotting on the struts.

In one aspect, the coating is configured to provide an appropriate bonding surface for a sensor wire routed to a distal portion of the hub body.

In one aspect of the disclosure is a method of forming a hub for an intravascular blood pump, the method comprising: placing a plurality of distal struts of the intravascular blood pump into a mold having a hub body portion and an arms portion; injecting one or more polymer materials while in molten form into the mold to form the hub, the hub having a hub body and a plurality of arms formed around the plurality of struts and extending radially away from the hub body.

In this aspect, the method may further comprise coupling the hub to the plurality of struts by overmolding the plurality of arms on the plurality of struts.

In this aspect, the one or more polymer materials may mold into engagement features of the plurality of struts to securely couple the plurality of arms with the plurality of struts.

In this aspect, the method may further comprise coupling the hub to the plurality of struts by inserting the plurality of struts into the distal openings of the plurality of arms, and using adhesive to bond the plurality of arms.

In this aspect, injecting the one or more polymer materials may include injecting a first polymer material to form the plurality of arms, and injecting a second polymer material to form the body.

In this aspect, the first and second polymer materials may be the same.

In this aspect, the first and second polymer materials may be different.

In this aspect, the first polymer material may be stiffer than the second polymer material.

In this aspect, the plurality of struts may be made of nitinol.

In this aspect, injecting the one or more polymer materials may comprise forming a distal tip of the intravascular blood pump such that the distal tip is integrally formed with the hub.

These and other details and aspects are described herein.

An intravascular blood pump is also provided, comprising: a collapsible conduit having an inner lumen for passing fluid therethrough, the conduit comprising a proximal section having a proximal opening, a distal section having a distal opening, and a central section positioned between the proximal and distal sections; at least one impeller within the conduit, the at least one impeller arranged to pump fluid into the distal opening of the conduit and out of the proximal opening of the conduit; a plurality of struts extending from the distal end of the conduit; a hub configured to receive the plurality of struts; and a distal atraumatic tip coupled to the hub; wherein a stiffness of the hub is greater than a stiffness of the distal atraumatic tip and the central section of the collapsible conduit, such that a load applied to the distal atraumatic tip is transferred to the central section of the collapsible conduit.

In one aspect, the hub comprises a plurality of arms extending radially away from a hub body, the plurality of arms being configured to receive the plurality of struts.

In this aspect, the plurality of struts extend into the hub body.

In one aspect, the blood pump may include a pressure sensor disposed in the distal hub.

In one aspect, the blood pump may include a guidewire lumen extending through the hub body and the distal atraumatic tip.

In one aspect the guidewire lumen includes a first portion having a first diameter in at least a portion of the hub body that is smaller than a second portion having a second diameter in the distal atraumatic tip.

In one aspect, a sidewall thickness of the hub body corresponding to the first portion of the guidewire lumen thicker than a sidewall thickness of the distal atraumatic tip corresponding to the second portion of the guidewire lumen.

In another aspect, the guidewire lumen tapers from the second diameter to the first diameter.

In one aspect, the tapering of the guidewire lumen is configured to prevent clots from exiting the guidewire lumen into the blood pump.

### BRIEF DESCRIPTION OF THE DRAWINGS

FIG. 1 is a side view of an exemplary blood pump that includes an expandable scaffold that supports a blood conduit with an impeller housed therein.

FIGS. 2A-2C show various parts of a blood pump showing an exemplary distal hub configured to promote smooth blood flow: FIG. 2A is a side view of a distal portion of the blood pump; FIG. 2B is a side view of the hub; and FIG. 2C is a front view of the hub.

FIGS. 3A-3D show the hub of FIGS. 2A-2C with a different blood conduit: FIG. 3A is a side view of a distal portion of the blood conduit; FIG. 3B is a side view of a scaffold of the blood conduit showing engagement features of the struts; FIG. 3C is another side view of the distal portion of the blood conduit; and FIG. 3D is a perspective view of the distal portion of the blood conduit.

FIGS. 4A-4C show alternative views of the hub of FIGS. 2A-2C and 3A-3D: FIG. 4A is a side view of the hub; FIG. 4B is a front view of the hub; and FIG. 4C is a section view of the hub.

FIGS. 5A and 5B show exemplary views of the hub of FIGS. 2A-2C, 3A-3D and 4A-4C in expanded and collapsed states: FIG. 5A is a perspective view of the hub and struts in an expanded state; and FIG. 5B is a perspective view of the hub and struts in a radially and torsionally collapsed state.

FIG. 6 shows a side view of an exemplary hub having two different compositions having different flexibilities.

FIGS. 7A-7C illustrate another embodiment of an exemplary hub of a blood pump.

FIG. 8 is a flowchart indicating an exemplary method of using a blood pump.

### DETAILED DESCRIPTION

The present disclosure is related to medical devices, systems, and methods of use and manufacture. In particular, described herein are pumps adapted to be disposed within a physiologic vessel, wherein the distal pump portion includes one or more components that act upon fluid. For example, the pumps herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

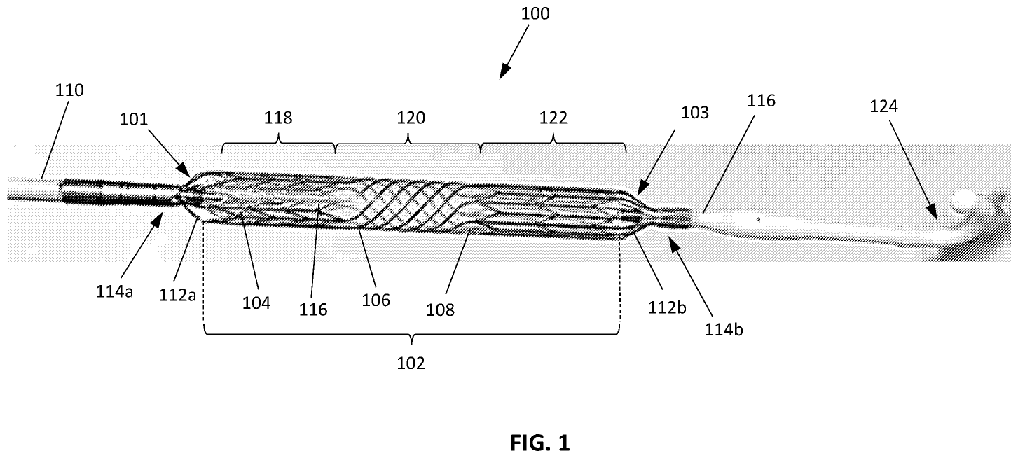

FIG. 1 shows a side view of an exemplary intravascular catheter blood pump 100. The blood pump 100 includes an expandable/collapsible blood conduit 102 that is configured to transition between an expanded state, as shown in FIG. 1, and a collapsed state (not shown). For example, the conduit 102 may be in the collapsed state when confined within a delivery catheter for delivery to the heart, expanded upon release from the delivery catheter for blood pumping, and collapsed back down within the delivery catheter (or other catheter) for removal from heart. When in the expanded state, the conduit 102 is radially expanded so as to form an inner lumen for passing blood therethrough. When in the expanded state, the inner lumen of the conduit 102 may be configured to accommodate blood pumped by one or more impellers therein. The one or more impellers may be collapsible so that they may collapse to a smaller diameter when the conduit 102 is in the collapsed state. The one or more impellers may be positioned within one or more impeller regions of the conduit 102. In some examples, the impeller region(s) of the conduit 102 is/are radially stiffer than other regions (e.g., adjacent regions) of the conduit 102 to prevent the impeller(s) from contacting the interior walls of the conduit 102.

In this example, the blood pump 100 includes an impeller 104 within a proximal portion of the conduit 102. In some cases, the blood pump 100 can include more than one impeller. For example, the blood pump 100 may include a second impeller in a distal region 122 of the fluid conduit 102. In some cases, blood pump 100 may include more than two impellers. The conduit 102 includes a first (e.g., proximal) end having a first (e.g., proximal) opening 101, and a second (e.g., distal) end having a second (e.g., distal) opening 103. The first opening 101 and second opening 103 may be configured as and an inlet and outlet for blood. For example, blood may largely enter the conduit 102 via the second (e.g., distal) opening 103 and exit the conduit 102 via the first (e.g., proximal) opening 101. In such case, the second opening 103 acts as a blood inlet and the first opening 101 acts as a blood outlet. The one or more impellers (e.g., impeller 104) may be configured to pump blood from the inlet toward the outlet. In an exemplary operating position, the second opening 103 (e.g., inlet) may be distal to the aortic valve, in the left ventricle, and the first opening 101 (e.g., outlet) may be proximal to the aortic valve (e.g., in the ascending aorta).

The exemplary conduit 102 includes a tubular expandable/collapsible scaffold 106 that provides structural support for a membrane 108 that covers at least a portion of inner surfaces and/or outer surfaces of the scaffold 106. The exemplary conduit is formed to be fluid impermeable by the membrane. The membrane may be attached to the scaffold, cover the scaffold, integrated into the scaffold, and other configurations as would be understood by one of skill from the description herein. The exemplary scaffold 106 includes a material having a pattern or plurality of openings with the membrane 108 covering the openings to retain the blood within the lumen of the conduit 102. The scaffold 106 may be unitary and may be made of a single piece of material. For example, the scaffold 106 may be formed by cutting (e.g., laser cutting) a tubular shaped material. Exemplary materials for the scaffold 106 may include one or more of: nickel titanium (nitinol), cobalt alloys, and polymers, although other materials may be used.

The exemplary scaffold 106 includes proximal struts 112a extending at a proximal end near the first opening 101 (e.g., blood outlet region) and distal struts 112b that extend from the scaffold 106 near the second opening 103 (e.g., blood inlet region). The proximal struts 112a are coupled to first hub 114a of a proximal shaft 110. The distal struts 112b are coupled to second hub 114b of a distal portion 114. In this example, the first hub 114a includes or is coupled to a bearing assembly through which a central drive cable 116 extends. The drive cable 116 is operationally coupled to and configured to rotate the impeller 104.

In some cases, the impeller 104 is fully positioned axially within the conduit 102. In other cases, a proximal portion of the impeller 104 is positioned at least partially outside of the conduit 102. That is, at least a portion of the impeller may be positioned proximally from a proximal end of the membrane 108. In some embodiments, the impeller 104 is partially positioned within the conduit and partially positioned within the first opening 101 (e.g., extending into the blood outlet region).

The conduit 102 and the scaffold 106 may be characterized as having a proximal region 118, a central region 120, and a distal region 122. The central region 120 may be configured to be placed across a valve (e.g., aortic valve) such that the proximal region 118 is at least partially within a first heart region (e.g., ascending aorta) and the distal region 122 is at least partially within a second heart region (e.g., left ventricle). The proximal region 118 (and in some cases the distal region 122) may be configured to house an impeller therein. The proximal region 118 may (and in some cases the distal region 122) have a stiffness sufficient to withstand deformation during operation of the blood pump 100 when within the beating heart and to maintain clearance (i.e., a gap) between an impeller region of the blood pump 100 and the rotating impeller 104. The distal region 122 includes the second (e.g., distal) opening 103 of the conduit 102, and may serve as the blood inlet for the conduit 102.

The central region 120 may be less rigid relative to the proximal region 118 (and in some cases the distal region 122). The higher flexibility of the central region 120 may allow the central region 120 to deflect when a lateral force is applied on a side of the conduit 102, for example, as the conduit 102 traverses through the patient's blood vessels and/or within the heart. For example, the central region 120 may be configured to laterally bend upon a lateral force applied to the distal region 122 and/or the proximal region 118. In some cases, it may be desirable for the central region 120 to laterally bend as the conduit 102 traverses the ascending aorta and temporarily assume a bent configuration when the conduit 102 is positioned across an aortic valve. In this example, the central region 120 includes a helical arrangement of longitudinally running elongate elements configured to provide flexibility for lateral bending while providing strength to prevent collapse of the blood conduit.

The blood pump 100 can further include a very flexible distal tip 124. In one implementation, the distal tip 124 is the most flexible component of the blood pump 100. In some embodiments, the distal tip can comprise a J-tip or a pigtail tip. In some examples, a distal tip 124 of the blood pump 100 is curved to form an atraumatic tip. In some cases, the distal tip 124 flexible (e.g., laterally bendable) to enhance the atraumatic aspects of the distal tip 124. For example, the distal tip 124 may be sufficiently flexible to bend when pressed against tissue (e.g., by a predetermined amount of force) to prevent puncture of the tissue.

The first hub 114a (e.g., proximal hub) and/or the second hub 114b (e.g., distal hub) may include features that promote smooth blood flow into and/or out of the conduit 102. Such features may prevent or reduce the occurrence of stagnant and/or turbulent blood flow that may otherwise tend to occur in regions near the first opening 101 (e.g., outlet region) and/or the second opening 103 (e.g., inlet region) of the conduit 102. Since stagnant and/or turbulent blood flow is associated with blood coagulation and/or clotting, measures to reduce this can be beneficial to for patient outcome.

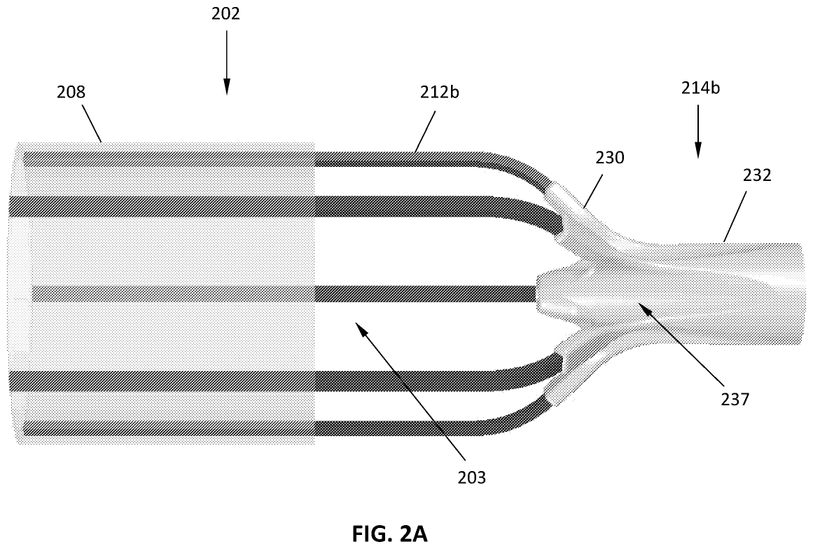

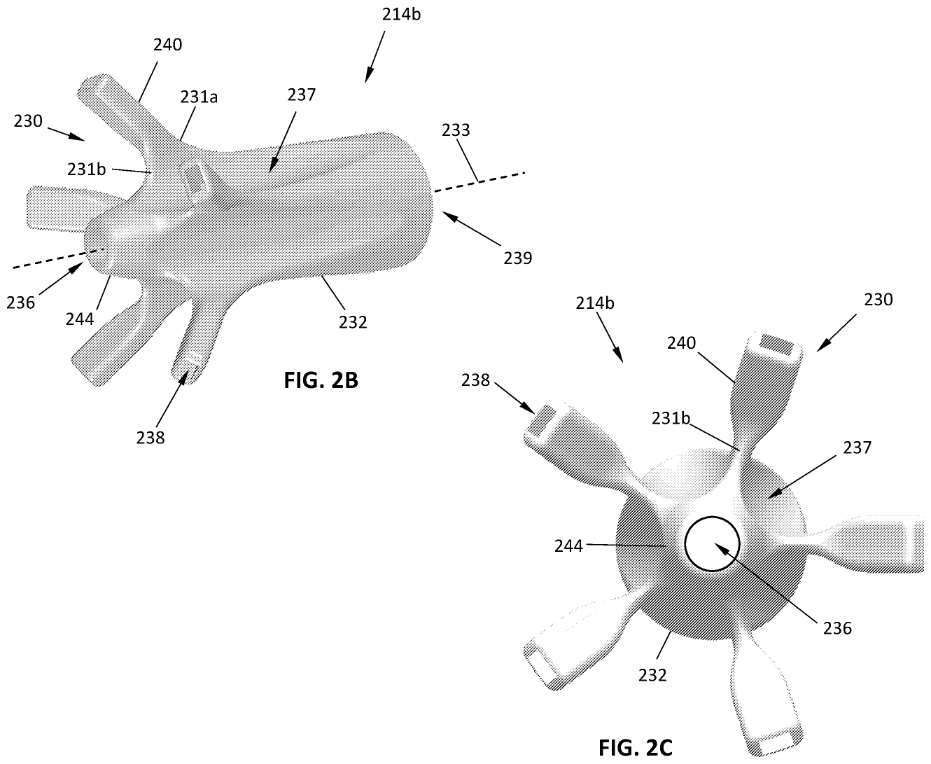

FIG. 2A-2C illustrate components of a blood pump with an exemplary distal hub 214b configured to reduce flow separation, recirculation, and/or stagnation past/through the hub and into the conduit. The distal hub is configured to prevent thrombus formation at the inlet of the blood pump by being completely smooth while limiting pockets/crevices or other areas near the inlet where blood cells can stagnate, collect, or attach to the blood pump. The distal hub can be designed and configured to prevent, reduce, or limit areas of stagnant blood flow, particularly under the distal struts of the scaffold, to prevent, reduce, or limit clot formation at or near the hub or struts. In general, all edges of the distal hub can be rounded to reduce separation/stagnation. FIG. 2A shows a side view of a distal end of a conduit 202, which includes a membrane 208 covering a portion of struts (e.g., distal struts) 212b that extend from the distal end of the conduit 202. FIGS. 2B and 2C show side and front views of the hub 214b. The hub 214b includes a body 232 with a number of arms 230, which extend radially from the body 232 and are coupled to the struts 212b. The number of arms 230 matches the number of struts 212b, which are coupled thereto. In this example, the hub 214b includes five arms that are about 72 degrees apart. However, the hubs described herein may include any number of arms (e.g., 1, 2, 3, 4, 5, 6, 7, 8, or more). For example, a hub may include 4, 5, 8, 10 or 16 arms. The arms may be equally spaced around the diameter of the hub, or have different spacings between the arms.

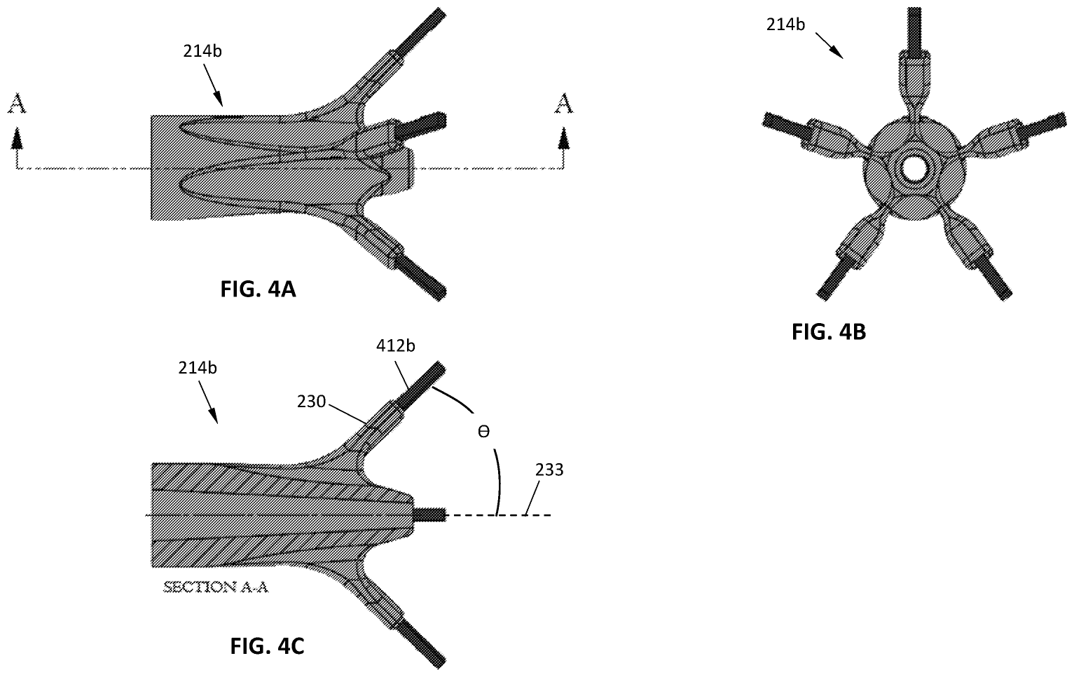

FIGS. 4A-4C shows various views of the hub 214b with exemplary dimensions according to some embodiments. FIG. 4C shows an angle θ (in this case 45 degrees) between a arm 230 (or strut 412b) and a central axis 233 of the hub 214b. In some cases, greater angles θ between the arms 230 and the central axis 233 may be associated with less blood stagnation. In some cases, the smaller angles θ may be associated with a higher occurrence of bridging between the arms 230 or struts 412b by cell growth. This may be balanced with concerns of crossing the anatomy. For example, if angle θ is larger (e.g., too close to 90 degrees), the blood pump may be more challenging to track into place, or potentially be traumatic to the anatomy. In some examples, the angle θ ranges between any of the following values: 20 to 90 degrees, 20 to 70 degrees, 20 to 60 degrees, 20 to 50 degrees, 30 to 90 degrees, 30 to 70 degrees, 30 to 60 degrees, and 30 to 50 degrees.

Returning to FIGS. 2A and 2B, the body 232 includes scalloped channels 237 that are shaped to direct fluid into the distal opening 203. The channels 237 can be convex or recessed into the main hub body, for example. In some cases, the channels 237 may be longer in an axial direction (e.g., along a central axis 233 of the hub 214b) than in a circumferential direction (e.g., around the body 232). The arms 230 have distal ridge regions 231a and proximal ridge regions 231b that extend axially along the body 235 to define the channels 237 and provide a smooth transition from the main hub body to the arms 230. The ridge regions 231a and 231b may be axially aligned with the central axis 233 of the hub 214b. Surfaces of the ridge regions 231a and 231b and/or the transition to channels 237 may be curved or smooth to reduce flow separation, recirculation, and/or stagnation around and past the hub 214b, struts 212b and the distal opening 203 of the conduit 202. The exterior surfaces of the hub 214b may be smooth to reduce friction from the fluid (e.g., blood) flow.

Each of the arms 230 includes a distal region 240 that extends from a corresponding ridge region 231a/b, and that is coupled to a corresponding strut 212b. From a side perspective (e.g., FIG. 2B), the portion of the arms defined by ridge regions 231a/b may be wider (e.g., in an axial direction) than the distal region 240 and gradually taper to the narrower distal region 240. From a front perspective (e.g., FIG. 2C), the distal region 240 may be wider (e.g., in a circumferential direction) than the ridge region 231 and gradually taper to the narrower region defined by ridge region 231a/b. This shape of the arms 230 may be configured to urge fluid in the channels 237.

In some embodiments, the ridge regions 231a and 231b may taper in the axial flow direction, as shown in FIG. 2C. Tapering the ridge regions is designed to promote fluid/blood flow along the channels and past the arms 230, and to prevent stagnant blood or reduce blood flow in that region of the distal hub. As shown, the ridge regions 231a and 231b may form a webbed base to arms 230, which can fill in spaces where blood may otherwise tend to stagnate and/or recirculate, thereby reducing or eliminating the occurrence of clot formation. The amount of space filled in between tapered feature 244 and arms 230 with ridge region 231b is carefully designed to reduce areas of stagnant blood flow but still allow for the flexibility required in the arms and struts to properly sheath and collapse the pump for delivery or removal.

In some cases, one or more portions of arms 230 may have a more extensive webbed structure. For example, in some examples, the ridge region 231b may extend further along a tapered feature 244 of the body 232 and the distal region 240 of one or more of the arms 230. In some cases, the ridge region 231b may extend to an extent that the ridge region 231b is coincident to an inner edge of the distal region 240 of the arm 230 and to a distal opening 238 (e.g., proximal opening) of the tapered feature 244, creating a more extensive webbing between the arms 230. The tapered feature 244, ridge region 231b, and arms 230 are designed and configured to more extensively fill in spaces where the blood may tend to accumulate, thereby further reducing or eliminating space for a clot to form. Additionally or alternatively, the hub 214b (e.g., the tapered feature and/or arms) is shaped and configured to promote desired blood flow through the inlet region of the pump. For example, the hub 214b may be shaped to reduce stagnant zones and/or reduce swirls. The hub 214b may be shaped to improve blood washing in desired locations.

The tapered feature 244 of the body 232 may also have a tapered shape conducive to smooth blood flow. The shape of the tapered feature 244 (e.g., diameter-to-length aspect ratio) may be optimizing to reduce blood stagnation and/or recirculation. In some embodiments, the tapered feature 244 extends proximally from a hub body, generally in the same location that the arms 230 extend radially (and proximally) away from the hub body. In some embodiments, the tapered feature 244 has an axial length that is shorter than, approximately equal to, or longer than an axial length of the arms 230. In some embodiments, the tapered feature can have a length of approximately 1-3 mm, a width or diameter of approximately 0.5-1.5 mm, and a tapering angle along its length of approximately 5-15 degrees.

Portions of the hub 214b may be integrally formed together. For example, the body 232 may be integrally formed with the arms 230. Some or all of the hub 214b may be formed by injection molding.

The distal region 240 of each of the arms 230 may have an opening 238 that is sized and shaped to accept and engage with a corresponding strut 212b. That is, the struts 212b may enter into the distal region 240 a corresponding strut 212b. In some cases, the arms 230 may be coupled to the distal end of a corresponding strut 212b using an overmolding process. For example, the one or more polymer materials, while in molten form, may be injected into one or more molds with the struts 212b positioned therein. Once the polymer material(s) harden, the arms 230 are formed and coupled to the struts 212b. In other cases, the struts 212b are positioned within already formed arms 230. An adhesive may be used to bond the struts 212b to the arms. In some cases, the struts 212b have engagement features that enhance the coupling of arms 230 with the struts 212b, such as described below.

The hub 214b may be made of any of a number of materials. In some examples, the hub 214b is made of a molded material, such as a polymer or urethane material. In some examples, the hub 214b is made of one or more of a polymer material and a metal material (e.g., shape memory metal). The material(s) may be suitably biocompatible for use in intravascular procedures. In some examples, the hub 214b may include radiopaque material to facilitate visualization of the tip features under fluoroscopy. In some cases, such radiopaque material may be doped/compounded into a polymer material as part of the hub 214b.

In some cases, at least a portion of the arms 230 are sufficiently flexible to bend radially inward when the conduit 202 is in a radially collapsed state. For example, when the conduit 202 is radially collapsed within a delivery catheter for delivery to the heart, the distal regions 240 of the arms 230 may be deflected radially inward toward the central axis 233 of the hub 214b. When the blood pump is released from the delivery catheter, the conduit 202 may radially expand outward (e.g., by force of shape memory material of the scaffold) along with the distal regions 240 of the arms 230. In some examples, at least the distal regions 240 of the arms 230 are made of a flexible material that preferentially assume the radially expanded state.

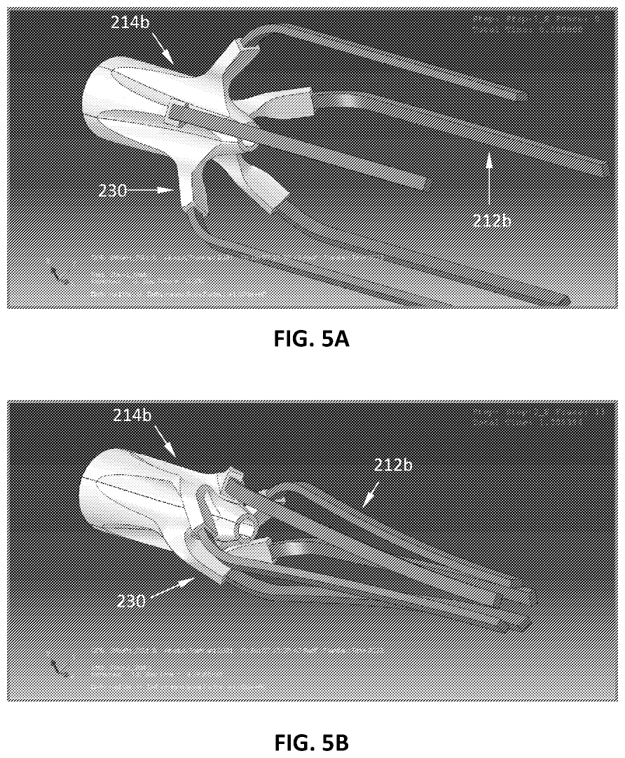

In addition to being radially collapsible, the arms 230 of the hub 214b (and/or the struts 312b of the scaffold) may twist to be torsionally collapsible. To illustrate, FIGS. 5A and 5B shows the exemplary hub 214b in a radially and torsionally expanded state (FIG. 5A) and in a radially and torsionally collapsed state (FIG. 5B). When in the radially and torsionally expanded state (FIG. 5A), the arms 230 and the struts 212b may radially expanded an in an untwisted configuration. When the conduit is transitioned to the collapsed state, the struts 212b and arms 230 may fold to the side, occupying the space between them, as shown in FIG. 5B. In some cases, the distal tip (e.g., 124, FIG. 1) may include a helical feature that causes the distal tip to preferentially twist in a similar manner. Such helical feature may follow a spiral pattern of the scaffold. This may also add benefit from a sheathing and flow direction aspect.

Returning to FIGS. 2B and 2C, in some cases, the body 232 of the hub 214b may include a first opening 236 (e.g., distal or proximal opening) and a second opening 239 (e.g., proximal or distal opening) that provide access to an inner lumen. In some examples, the lumen extends to the distal end of the distal tip (e.g., 124, FIG. 1) of the blood pump, where they may or may not be a septum. The lumen may be shaped and sized to accommodate a guidewire. The guidewire may enable a physician to push the blood pump through the anatomy and into place across the heart valve. The lumen on the distal tip may enable this and help steer the blood pump through the anatomy.

In some cases, the lumen within the body 232 of the hub 214b may accommodate a shaft (e.g., driveshaft) that is operationally coupled to one or more impellers of the blood pump. In some cases, the lumen may house a bearing assembly, which may be operationally coupled to the one or more impellers of the blood pump. In some cases, the hub 214b does not include a bearing assembly.

The hub 214b may be coupled to an atraumatic distal tip (e.g., 124, FIG. 1) of the blood pump. In some examples, the hub 214b may be integrally formed with the distal tip. For example, the hub 214b may smoothly transition to the distal tip. In some cases, the hub 214b may be made of the same material(s) as the distal tip. In other embodiments, the hub and the tip are separate components that are later bonded, attached, or adhered together.

In some examples, a wall thickness (durometer) along the length of the hub 214b and/or distal tip (e.g., 124, FIG. 1) may vary to aide in flexibility of certain regions.

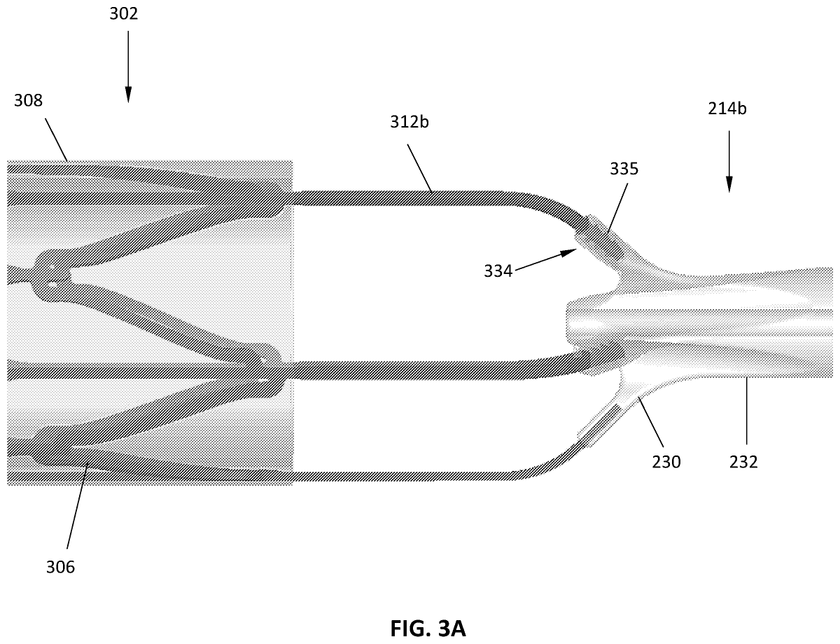

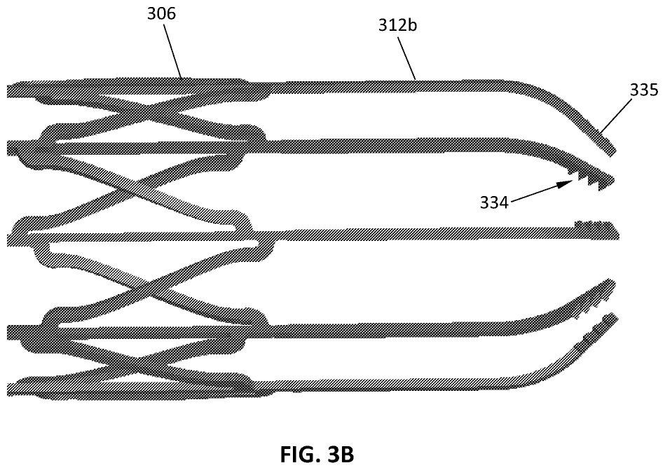

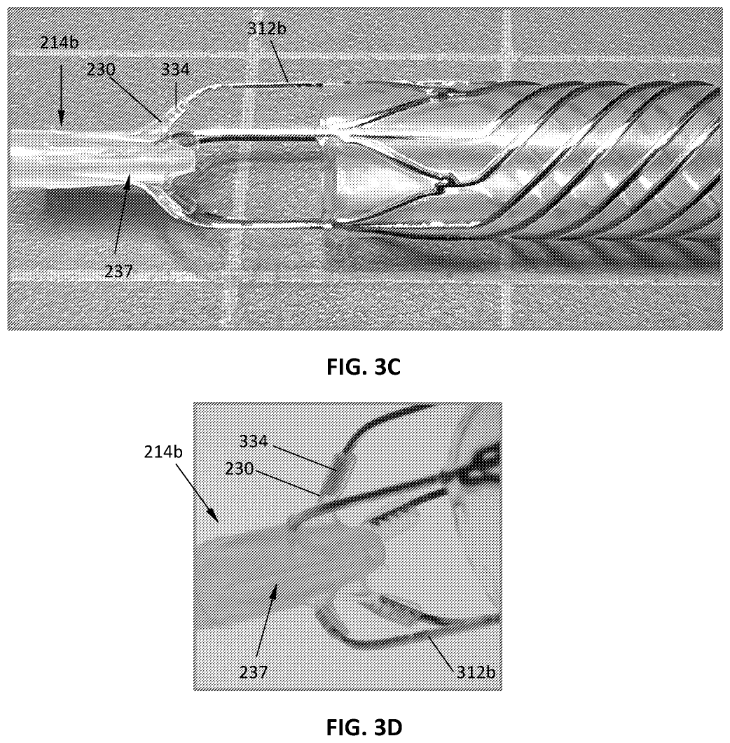

FIGS. 3A-3D illustrate components of another exemplary conduit 302 connected with the distal hub 214b. In this case, the conduit 302 includes a membrane 308 that covers the scaffold 306 but largely does not cover the struts 312b. As described above, the struts 212b may be integrally formed with and be made of the same material as the scaffold 306. FIG. 3A illustrates a semitransparent view of the hub 214b, showing distal ends 334 of the struts 312b within the arms 230. FIG. 3B illustrates the distal ends 334 of the struts 312b without the hub 214b coupled thereto. FIGS. 3C and 3D shows additional views of the hub 214b and conduit 302. As shown, the distal ends 334 of the struts 312b may include engagement features 335 that may enhance coupling with a corresponding arm 230. The engagement features 335 may include barbs, serrations, holes, protrusions, recesses, or other geometries shaped to increase the bond strength between the struts 312b and the arms 230, thereby increasing the pull-out force required to separate the struts 312b and the hub 214b. In some cases, one or more bonding agents (e.g., epoxy or glue) may be used to further strengthen the attachment. In some cases, a surface treatment of the struts 312b (e.g., nitinol surface treatment) may be implemented to increase the bond strength. Such surface treatments may include one or more surface abrasion, plasma etching, plasma cleaning and/or polishing treatments.

In some cases, the hub 214b is coupled to the struts 312b by overmolding the arms 230 on the struts 312b. For example, the one or more polymer materials, while in molten form, may be injected into one or more molds with the struts 312b positioned therein. The molten polymer material(s) may conform to the shape of the engagement features 335 of the struts 312b, thereby forming correspondingly shaped engagement features within the opening of the arms 230 once the molten polymer material(s) harden. In other cases, the struts 312b are positioned within already formed arms 230. An adhesive may be used to bond the struts 312b to the arms.

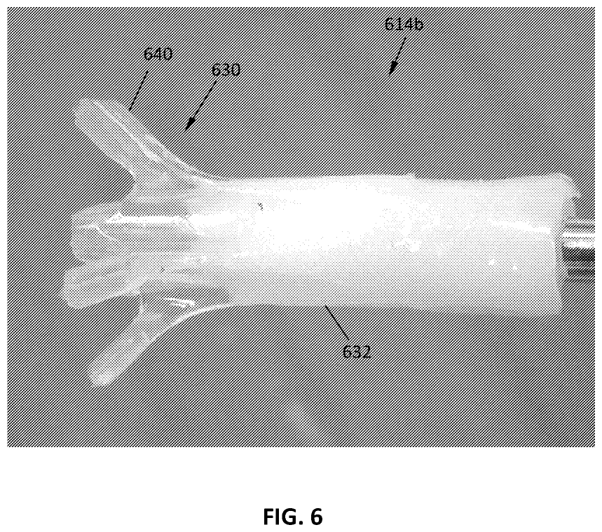

In any of the blood pumps described herein, the hub may have regions of different compositions. For example, two or more different polymer materials (e.g., having different stiffness durometers) may be molded together to form different regions of the hub. FIG. 6 shows an example of a hub 612b having regions of two different compositions. In this example, at least a portion of the distal regions 640 of the arms 630 is made of a first material and at least a portion of the body 632 is made of second material that is different than the first material. In some examples, the first material forming at least a portion of the distal regions 640 of the arms 630 is stiffer (e.g., having a stiffer durometer) material to facilitate retention and reduce tear out risk of the struts. The body 632 of the hub 614b may be made of a less stiff (e.g., softer durometer) material, for example, to match the compliance of the distal tip. In some cases, the different materials may have different colors (e.g., clear distal regions 640 and white body 632).

In any of the blood pumps described herein, the geometry in the distal tip (e.g., 124) may be adapted to optimize a retention force, radial strength, recovery from collapsed state, and/or vibration isolation (e.g., for pressure sensor(s) accuracy).

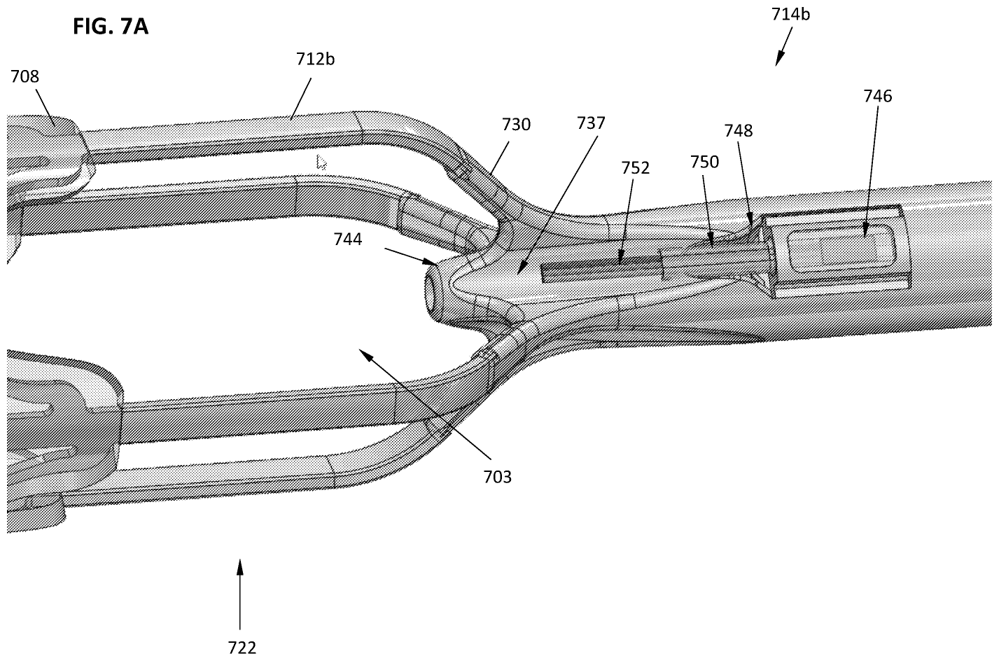

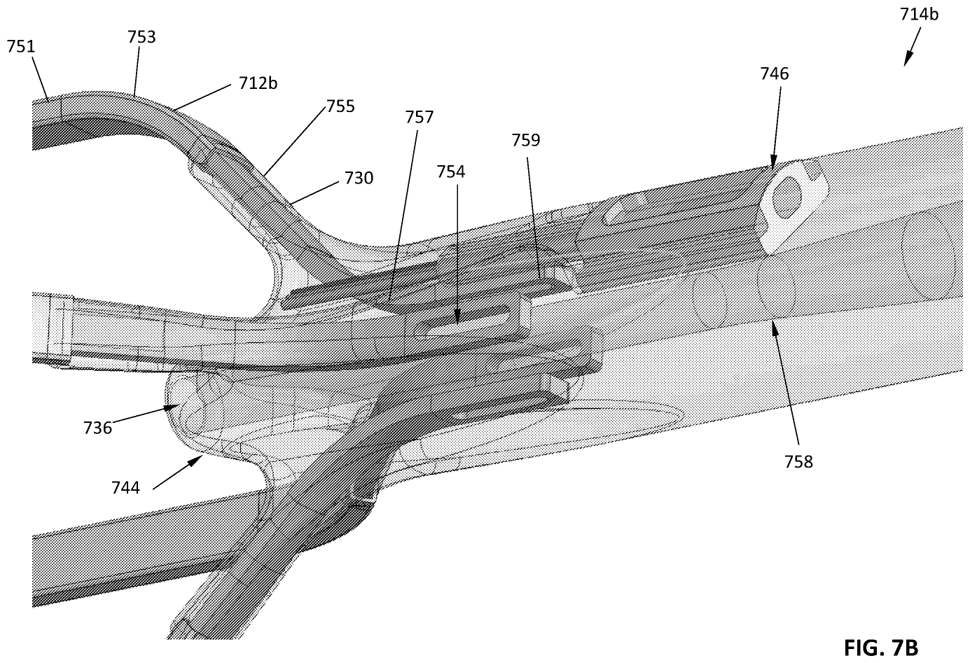

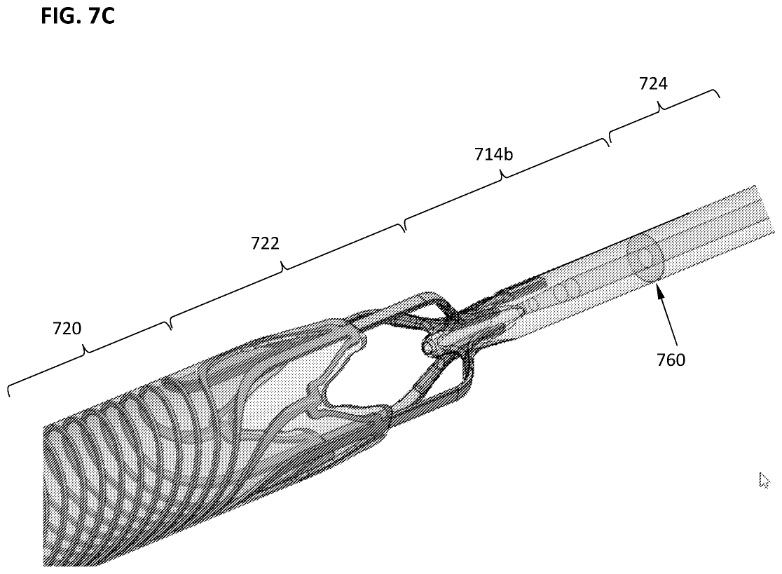

FIG. 7A illustrates another embodiment of a blood pump distal hub 714b which may include may of the features previously described, including arms 730 configured to receive struts 712b, channels 737, and tapered feature 744. As previously described, the hub, struts, and membrane 708 collectively form the distal portion 722 of the blood pump including the distal opening 703 (e.g., the inlet).

In any of the blood pumps described herein, the hub and/or struts may include routing features to accommodate the routing of a sensor (e.g., pressure sensor), wire(s), or fiber(s) to the distal end of the blood pump. In some cases, such routing features are arranged to route the wire(s) and/or fiber(s) along one or more of the arms and/or struts to traverse the conduit. FIG. 7A shows the incorporation of a distal pressure sensor 746 into the hub 714b. The pressure sensor can comprise, for example, a MEMS pressure sensor or any other pressure sensor as known in the art. As shown the hub can include a cutout or recessed portion 748 configured to receive the pressure sensor. The pressure sensor can be glued, bonded, heat treated, or snap fit into the recessed portion 748 of the hub. In some embodiments, a separate sensor housing is used to receive the sensor, and the sensor and sensor housing are then collectively inserted into the cutout. The hub can also include strain relief features 750 extending from the sensor or the sensor housing, to allow for bending and routing of the sensor wires. In some embodiments, the sensor wires are routed along the arms 730, along struts 712b, and then routed either along the scaffold (inside or outside) or covered with the membrane 708 as they extend proximally along the device.

In some embodiments, the struts 712b are covered with a polymer material to reduce friction/rough edges along the section of struts that are exposed between the membrane 708 and the arms 730. For example, after laminating the shroud with the membrane 708, polymer tubing can be placed over the struts, which can then be encapsulated by the hub. The polymer coating over the struts 712b provides a better bond between the sensor wires and the struts, as bonding the sensor wires directly to bare metal or nitinol would be less effective. Additionally, when the device is collapsed or compressed, the struts can come into contact with the guidewire exiting the tapered feature 744. The polymer coating over the struts 712b prevents damage to the guidewire (often Teflon coated) when the blood pump is collapsed or in a delivery state. Uncoated struts, typically made of a metal such as nitinol, can include rough spots or areas that may otherwise damage the guidewire if uncoated, and may also provide micro abrasions or rough spots on the struts that could cause clot formation. Coating the struts with the polymer or other smooth coating can therefore prevent clot formation on the struts.

FIG. 7B illustrates a semi-transparent view of the hub 714b of FIG. 7A. In this view, it can be seen how the distal ends 734 of struts 712b extend past the arms 730 and into the hub body itself. In this example, the distal ends 734 of struts 712b extend almost to the distal pressure sensor 746. As shown in FIG. 7B, each of the struts 712b can include a first straight section 751 extending distally away from the conduit, a first curved section 753 that transitions into a second straight section 755 into the arms 730, and a second curved section 757 that transitions into a straight end section 759. Extension of the struts into the hub body itself, instead of terminating the struts within the arms in the designs described above, can prevent buckling or bending at the strut/arm transition when large forces are applied to the blood pump. The straight end section 759 of the struts can further include slits or through features 754. As previously described, the scaffold of the blood pump is laminated with the membrane to form the blood conduit, and the struts 712b can be covered with polymer (e.g., polymer tubing) and placed in the hub mold. Then one or more polymer materials can be injected into the hub mold while in molten form to form the hub. The slits or through features 754 allow for flow-through of the polymer material(s) during injection molding to ensure a strong bond between the struts and the hub and to prevent the struts from withdrawing from the hub.

FIG. 7B also shows guidewire lumen 756 which extends from opening 736 in the tapered feature, through the hub 714b, and through the distal tip (e.g., distal tip 124). As shown in FIG. 7B, the guidewire lumen can have a variable diameter along its length. In this example, the guidewire lumen has a first diameter along the section of guidewire lumen extending from the opening 736 to junction 758, and then the guidewire lumen expands to a second diameter past the junction 758 (optionally along the remainder of the guidewire through the distal tip (e.g., tip 124). Varying the diameter of the guidewire lumen allows for adjustment of the stiffness of the hub 714b. For example, since the first diameter between opening 736 and junction 756 is smaller, the hub has a larger sidewall thickness in that region, providing more stiffness in the hub in that region. According to one embodiment, this region of decreased guidewire diameter (and increased hub wall thickness) can be aligned with the sections of the hub that include the struts 712b, the opening/inlet 703, and the pressure sensor 746. Generally it is desirable to have this section of the hub stiffer than the neighboring sections of the blood pump to avoid kinking or bending in this region of the pump. Furthermore, increasing the diameter of the guidewire past the junction 756 serves to reduce guidewire friction in that region of the tip. Additionally, the narrowed or tapered section of guidewire lumen can also serve to trap clots or blood that collects in the guidewire lumen during a procedure, thereby preventing thrombus or clots from entering the pump from the guidewire lumen.

FIG. 7C is another view of a blood pump, including a central region 720, a distal region 722, a distal hub 714b, and a distal tip 724. It is noted that the full distal tip 724 is not shown, but should be understood that this distal tip can extend further in the distal direction and terminate as a J-tip, pigtail tip, or other atraumatic tip (as depicted in FIG. 1). Junction 760 is shown in FIG. 7C, which is the junction between the distal hub 714b and the distal tip 724. In some embodiments, the distal hub and the distal tip are bonded, adhered, connected, or heat treated to form the connection between the hub and tip. In some embodiments, the length of the hub is approximately ¾″ to 1″ in length. This length provides the space needed in the hub to house the strut distal ends, form the tapered feature and arms, and provide a stiff and secure location for the distal pressure sensor that is spaced apart from the inlet so as to avoid turbulent areas of blood flow. Providing this spacing between the hub/tip junction also allows for heating/bonding of the tip to the hub after the hub itself has been heat treated/formed, without re-melting or adversely affecting the previously formed hub components.

The various sections within the blood pump have been designed with specific stiffness/load characteristics to allow for loads applied to the blood pump to be transferred to desired regions within the pump and avoid bending/kinking in other regions within the pump. Generally, referring to FIG. 7C, the most flexible/least stiff regions of the blood pump can be the distal tip 724 (including the J-tip, pigtail, or curved portion at the very distal end) and the central region 720 (e.g., the region of the conduit/shroud that includes the helical shroud pattern). In this embodiment, the distal region 722 and hub 714b are stiffer than the central region and distal tip. This allows for a load applied to the distal tip (e.g., contacting the heart wall with the distal tip) to be transferred from the distal tip to the center of the shroud (central region 720) to prevent bending at the hub or inlet of the pump.

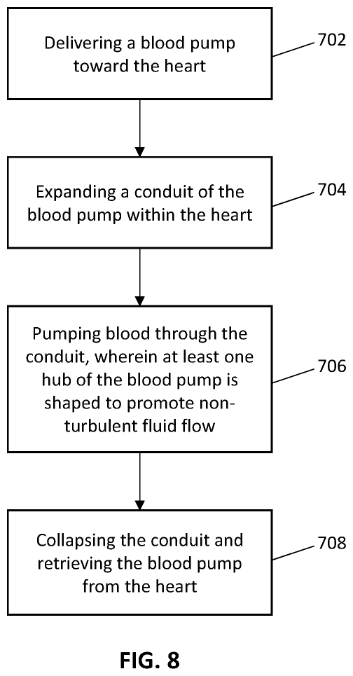

FIG. 8 is a flowchart illustrating an exemplary method of using an intravascular blood pump. At 702, the blood pump is delivered to the heart. The blood pump may be in a collapsed state, for example within a delivery catheter, to advance the blood pump through vessels of the patient and to a target area of the heart. In some cases, the blood pump is positioned within a valve (e.g., aortic valve) such that the distal opening of the blood conduit and a distal hub are within the ventricle, and the proximal opening of the blood conduit and a proximal hub are within the atrium and/or aorta (e.g., ascending aorta).

At 704, a conduit of the blood pump is expanded within the target area of the heart. The conduit may be expanded by releasing the blood pump from the delivery catheter. The conduit may include a scaffold made of a shape memory material that preferentially assumes an expanded state. Expanding the conduit may cause arms of the distal hub and/or proximal hub, which are connected to conduit, to radially expand from a collapsed state.

At 706, blood is pumped through the conduit by causing at least one impeller within the conduit to rotate. The proximal hub and/or the distal hub may include a bearing assembly that is/are operationally coupled to the at least one impeller within the conduit. In some examples, the blood pump is configured to pump blood into the distal end of the conduit and out of the proximal end of the conduit. The distal and/or proximal hub may include channels shaped to reduce flow separation, recirculation, and/or stagnation into the distal opening and/or out of the proximal opening of the conduit. The channels may extend axially along a body of the distal and/or proximal hub. The arms of the distal and/or proximal hub may include ridge regions that define the channels.

At 708, the conduit is collapsed, and the blood pump is retrieved from the heart. The conduit may be collapsed into a radially collapsed state by retracting the intravascular blood pump into a catheter. Collapsing the conduit may cause the arms to radially collapse from the expanded state. Once blood pump is in the collapsed shape (e.g., within the catheter), the blood pump may be retrieved from the heart by pulling the blood pump out of the patient's blood vessels.

Although FIGS. 2A-2C, 3A-3D, 4A-4C, 5A-5B, and 7A-7C show examples a distal hub that is distal to the blood conduit, in some cases, a proximal hub having similar flow promoting features (e.g., channels) may be positioned proximal to the conduit. For example, a proximal hub may have a body and arms shaped to form channels that reduce flow separation, recirculation, and/or stagnation out of a proximal opening (e.g., outlet) of the conduit. Such proximal hub may be used with or without the distal hub shown in FIGS. 2A-2C, 3A-3D, 4A-4C 5A-5B, and 7A-7C.

Any of the blood pumps described herein may include surfaces with one or more anticoagulant agents. For example, at least a portion of one or more of the hubs, conduits (e.g., scaffold and/or membrane), struts (e.g., proximal and/or distal struts), distal tips and/or impellers of the blood pumps described herein may include a coating or material having an anticoagulant agent. In some cases, the anticoagulant agents may include drugs such as heparin, warfarin and/or prostaglandins.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

1. A hub for an intravascular pump, comprising:
a hub body; and
a plurality of arms radially extending from the body, wherein the plurality of arms each have an opening sized and configured to receive a strut of a scaffold structure of the intravascular pump.

2. The hub of claim 1, wherein the plurality of arms are comprised of a polymer material.

3. The hub of claim 1, wherein the hub body is comprised of a polymer material.

4. The hub of claim 1, wherein the hub body and the plurality of arms are comprised of a same polymer material.

5. The hub of claim 1, wherein the hub body and the plurality of arms are comprised of different polymer materials.

6. The hub of claim 5, wherein the plurality of arms are made of a stiffer polymer than the hub body.

7. The hub of claim 1, wherein the hub body is integrally formed with the plurality of arms.

8. The hub of claim 1, wherein the hub is formed by injection molding.

9. The hub of claim 1, wherein each of the arms has a distal opening with engagement features configured to engage with a distal end of a corresponding strut.

10. The hub of claim 1, wherein the hub is integrally formed with a distal tip of the intravascular pump, the distal tip having an atraumatic distal end.

11. The hub of claim 1, wherein the hub is bonded to a distal tip of the intravascular pump, the distal tip having an atraumatic distal end.

12. The hub of claim 1, wherein each of the plurality of arms has a webbed base that extends axially along the hub body to define channels between adjacent arms.

13. The hub of claim 12, wherein the webbed bases of the plurality of arms form channels along the hub body of the hub between the plurality of arms.

14. The hub of claim 1, further comprising a tapered feature extending proximally from the hub body.

15. The hub of claim 12, wherein the webbed bases transition from each of the plurality of arms to a tapered feature that extends proximally from the hub body.

16. The hub of claim 1, wherein the hub body has a lumen that extends axially through the hub body.

17. The hub of claim 15, wherein the hub body has a lumen extends axially from the tapered feature through the hub body.

18. The hub of claim 1, further comprising a pressure sensor disposed within the hub body.

19. The hub of claim 18, further comprising at least one pressure sensor wire routed proximally from the pressure sensor along at least one of the plurality of arms.

20. An intravascular blood pump, comprising:
a collapsible conduit having an inner lumen for passing fluid therethrough, the conduit comprising a proximal end having a proximal opening, and a distal end having a distal opening;
at least one impeller within the conduit, the at least one impeller arranged to pump fluid into the distal opening of the conduit and out of the proximal opening of the conduit;
a plurality of struts extending from the distal end of the conduit; and
a hub comprising a hub body with a plurality of arms extending radially from the hub body, the plurality of arms each have an opening sized and configured to receive one of the plurality of struts.

21.-71. (canceled)
